(19) United States
(12) Patent Application Publication
(10) Pub. No.: US 2021/0275767 A1
(43) Pub. Date: Sep. 9, 2021

(54) A DEVICE FOR HOLDING ENDOTRACHEAL TUBES

(71) Applicant: CLIP FAB R&D MEDICAL INSTRUMENTS, Barcelona (ES)

(72) Inventors: Antonio FERRANDIZ CATALAN, Barcelona (ES); Jose Miguel CASANOVAS RODRIGUEZ, Barcelona (ES)

(73) Assignee: CLIP FAB R&D MEDICAL INSTRUMENTS, Barcelona (ES)

(21) Appl. No.: 16/348,738

(22) PCT Filed: Nov. 21, 2016
(86) PCT No.: PCT/ES2016/070829

§ 371 Date: May 9, 2019

(51) Int. Cl. A61M 16/04

(52) U.S. Cl. CPC A61M 16/0493; A61M 2025/022

(57) ABSTRACT

A device for holding endotracheal tubes includes a bite tube that defines a passage zone for at least one endotracheal tube; a front plate and an adjustable retainer joined to the bite tube, and which form means for limiting displacement of the bite tube in an axial direction towards the interior and exterior of the mouth of the patient, respectively; and means for immobilising at least one endotracheal tube with respect to the bite tube in an axial direction. The device includes a lateral opening along the entire length of the bite tube, the front plate and the adjustable retainer for lateral or transversal introduction of at least one endotracheal tube into the passage zone defined by the bite tube.

### OBJECT OF THE INVENTION

This invention relates to a device for holding endotracheal tubes, intended to secure at least one endotracheal tube with respect to a patient's mouth and comprising: a bite tube that defines a passage zone for at least one endotracheal tube; a front plate and an adjustable retainer, preferably inflatable, joined to the bite tube and forming means to limit displacement of the bite tube in an axial direction towards the interior and exterior of the mouth of the patient, respectively, and in a transversal direction; and means for immobilising at least one endotracheal tube with respect to the bite tube in an axial and transversal direction.

This holding device presents characteristics aimed at facilitating its fitting over the endotracheal tube or tubes, both in an axial and a transversal direction.

### SCOPE OF THE INVENTION

This invention is relevant to hospitals, mobile healthcare units, emergency care and medical or veterinary care in general.

### PRIOR ART

Currently, catheters and endotracheal tubes are widely used in the medical health field for different purposes such as to provide oxygen or liquids to the body or eliminate secretions from inside the body.

Once the patient has been intubated correctly, the endotracheal tube needs to be held to prevent it from moving forwards inadvertently into or out of the patient.

Traditionally, the endotracheal tube is held in place with respect to the patient using straps encircling said tube, outside the patient's mouth and fixed behind the patient's head or neck.

As well as being awkward, slow and insecure, this holding method is impractical as it prevents or at least significantly obstructs other operations such as cleaning or treating the patient's mouth; as well as impairing the appearance of the intubated patient.

Another disadvantage is that the endotracheal tube, which passes through the interior of the patient's mouth, can be bitten by the patient themselves causing its obstruction and the consequent loss of functionality of the endotracheal tube.

These problems were solved in part by the device described in document US 20020095118 A1 (WO2001091838 A1) which comprises: a tubular-shaped bite block with a distal end and a proximal end; an inflatable balloon attached to the distal end of said bite block and a shield extending from the proximal end of said bite block.

This tubular-shaped bite block is intended to be introduced into the patient's mouth 20 and to define a passage closed at the perimeter for the introduction of the endotracheal tube; said bite block acts as a protection of the endotracheal tube preventing the patient from being able to bite or obstruct it.

The inflatable balloon located on the distal end of the bite block has a dual function—on the one hand, it is positioned inside the patient's mouth behind their teeth so preventing the displacement of the device towards the outside of the mouth; and, on the other, it exerts pressure on the endotracheal tube that passes through the interior of the bite block, preventing the axial displacement of the endotracheal tube 30 with respect to the holding device.

This prior art also provides for the device to be able to incorporate two inflatable balloons to perform the two previous functions separately; in both cases, the inflatable balloon or balloons have one inflation line from the exterior of the patient's mouth.

This device presents specific disadvantages for use in terms of the ease of its fitting and removal.

Given that the block or bite tube has only one axial orifice for fitting it around the endotracheal tube or tubes, it has to be assembled in the axial direction by introducing one of the ends of said tubes via said axial orifice; furthermore, once the patient has been intubated, the endotracheal tube must be held in the correct position with one hand and the balloon or balloons that secure the device with respect to the patient's mouth in the direction of removal must be inflated, and the endotracheal tube secured with respect to the device, with the other hand.

Despite the existence of said prior art, there is a need to develop a device for securing endotracheal tubes with respect to a patient's mouth that simplifies the fitting and removal of the device with respect to the endotracheal tube or tubes, once the patient has been intubated.

### DESCRIPTION OF THE INVENTION

The endotracheal tube holding device that is the object of this invention is intended to secure at least one endotracheal tube with respect to a patient's mouth and is of a type that comprises: a bite tube that defines a passage zone for at least one endotracheal tube; a front plate and an adjustable retainer, preferably inflatable, joined to the bite tube and which form means for limiting displacement of the bite tube in an axial direction towards the interior and exterior of the mouth of the patient, respectively, and in a transversal direction; and means for immobilising at least one endotracheal tube with respect to the bite tube in an axial and transversal direction.

This device has special design features aimed at solving the problems set out above satisfactorily and facilitating its fitting and removal with respect to the endotracheal tube once the patient has been intubated, so that it can be removed easily, for example to treat or clean the patient's mouth, without the need to extubate the patient or disconnect the endotracheal tube from any external device, for example, for supplying liquid or oxygen to the patient.

To achieve the objectives proposed, this device has the special feature of a lateral opening along the entire length of the bite tube, the front plate and the adjustable retainer for lateral or transversal introduction of at least one endotracheal tube into the passage zone defined by the bite tube.

The front plate and adjustable retainer are arranged externally with respect to the bite tube so immobilising the device with respect to the patient's mouth only, as it is attached via the pressure exerted by the adjustable retainer when inflated, preferably on the internal part of the teeth, trapping the teeth between the adjustable retainer, preferably, and the front plate.

When in position for use, the front plate is adjusted between the patient's teeth and lips to prevent lip blisters or sores.

The means of immobilisation of the endotracheal tube comprise a pressure clamp attached to the front plate, open towards the lateral opening of the device and arranged in parallel to an exterior face of the front plate.

According to the invention, these characteristics allow the device to be assembled and disassembled in the transversal or lateral direction, on any intermediate point of the endotracheal tube, simply by introducing said tube via a lateral opening of the device, defined for this purpose on the bite tube, the adjustable retainer, the front plate and the pressure clamp.

This characteristic simplifies the fitting and removal of the device with respect to the endotracheal tube or tubes considerably, once the patient is intubated, as the ends of said tubes do not need to be free or accessible.

According to the invention, the pressure clamp that is placed parallel to an exterior face of the front plate comprises an end part attached to this front plate and two elastically flexible arms, which define toothed concave portions, facing each other, to secure at least one endotracheal tube in the passage zone of the bite tube.

This front plate comprises stops for securing the arms of the pressure clamp, elastically bent into an open position of this clamp; this enables the device to be fitted on any intermediate area of the endotracheal tube or tubes and moved in an axial direction with respect thereto, for example, during the correct positioning of the device with respect to the patient's mouth, maintaining the clamp in an open position.

For the pressure clamp to be in an operational position, its arms simply need to be released from the retaining stops so that they then exert pressure on the endotracheal tube or tubes passing through the interior of the device, preventing its relative displacement in an axial direction.

Therefore, said pressure clamp makes it possible to hold and release the endotracheal tube or tubes that pass through the interior of the device, rapidly and simply, without the need to use or manipulate external elements such as an inflation tube or pressure syringe operated manually.

The design of the holding clamp may vary according to its adaptation to different endotracheal tube sizes.

### DESCRIPTION OF THE FIGURES

To supplement this description and facilitate the understanding of the characteristics of the invention, this description is accompanied by a set of drawings in which the following have been represented with an illustrative rather than limitative character:

FIG. 1 is a front perspective view of one embodiment of the device for holding endotracheal tubes according to the invention.

FIG. 2 is a back elevation view of the holding device in the previous figure.

FIG. 3 is a front elevation view of the holding device in the previous figures.

FIG. 4 is a top plan view of the holding device in the previous figures divided by the horizontal plane shown in FIG. 3.

FIG. 5 is a bottom plan view of the holding device in the previous figures with an adjustable retainer, constituted by an inflatable balloon, shown in a deflated position.

FIG. 6 is a front elevation view of the holding device in the previous figures during the introduction of an endotracheal tube in the transversal direction, in its interior and with the pressure clamp held in an open position.

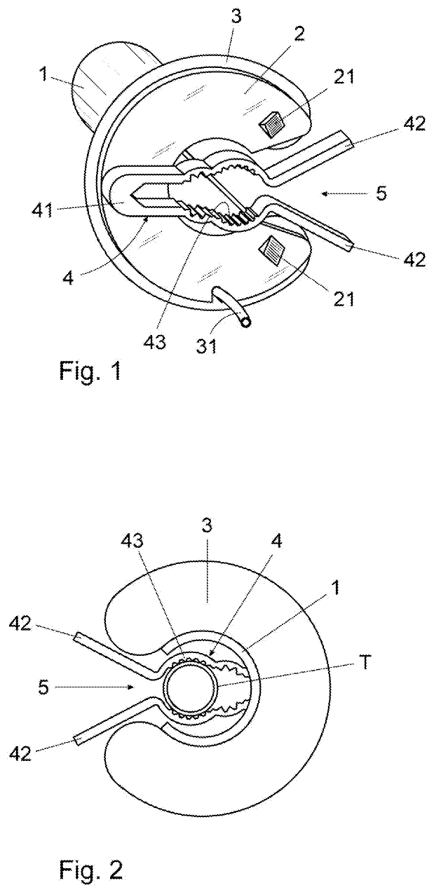

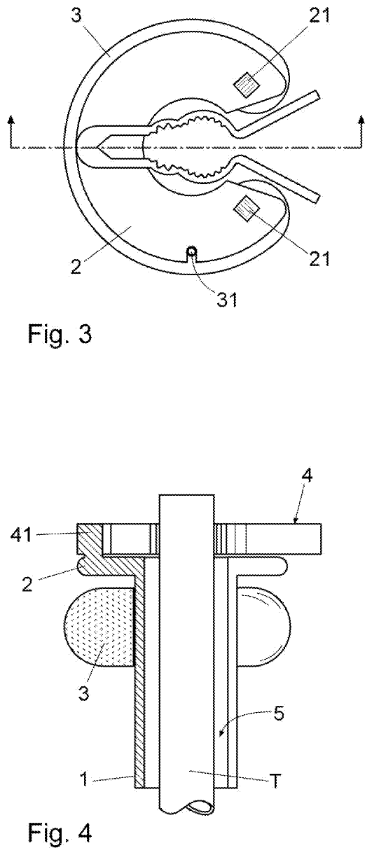

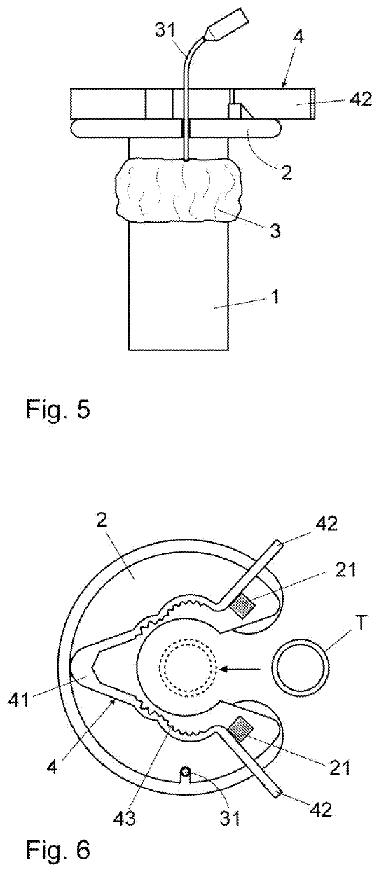

### PREFERRED EMBODIMENT OF THE INVENTION

In the embodiment example shown the attached figures, the device for holding the endotracheal tubes comprises: a bite tube (1); a front plate (2) and an adjustable retainer (3) arranged externally with respect to the bite tube (1) and substantially perpendicular to said bite tube (1); and a pressure clamp (4) attached to the front plate (2).

In the example shown, the adjustable retainer (3) is constituted by an inflatable balloon.

This inflatable balloon (3) is intended to be positioned and inflated inside the patient's mouth, trapping the device against the patient's teeth, preventing the device from moving in an axial direction towards the exterior of the mouth; whilst the front plate (2) is intended to be positioned between the patient's teeth and lips, preventing the axial displacement of the device towards the interior of the mouth.

As can be observed in the attached figures, this device comprises a lateral opening (5) along the entire length of the bite tube (1), the front plate (2) and the inflatable balloon (3) for lateral or transversal introduction of at least one endotracheal tube (T) into the passage zone (11) defined by the bite tube (1), as shown in FIG. 6.

The inflatable balloon (3) has an inflation tube (31) for the connection of a pressure syringe that can be actuated manually or any other suitable device for supplying air or fluid under pressure and the inflation thereof.

The pressure clamp (4) is arranged in parallel to an exterior face of the front plate (2) and comprises: an end part (41) attached to this front plate and two elastically flexible arms (42), which define toothed concave portions (43), facing each other, to secure at least one endotracheal tube (T) in the passage zone (12) of the bite tube (1) as shown in FIG. 2.

The front plate (2) comprises stops (21) for securing the arms (42) in the open position of the clamp (4) represented in FIG. 6.

These stops (21) have ramps oriented towards the lateral opening (5) of the device to facilitate the positioning of the arms (42) of the pressure clamp (4) in the open position, it being sufficient to separate the arms (42) in the direction of the opening of the clamp (4) until they pass said stops 21 and are held in the open position represented in FIG. 6 for this purpose.

To return the clamp (4) to the operational position represented in FIG. 2, the arms (42) simply need to be bent slightly in the front direction for them to be released from the stops (21) and return to said operational position as a result of the action of the elastic energy accumulated therein.

Once the nature of the invention has been described sufficiently, along with a preferred embodiment example, it is noted that the materials, shape, size and arrangement of the parts described may be altered for the appropriate purposes provided that this does not change the essential characteristics of the invention claimed below.

1. A device for holding endotracheal tubes, intended to secure at least one endotracheal tube with respect to a patient's mouth and comprising:
a bite tube that defines a passage zone for at least one endotracheal tube,
a front plate and an adjustable retainer joined to the bite tube and which form means for limiting displacement of the bite tube in the axial direction towards the interior and exterior of the mouth of the patient respectively; and
means for immobilising at least one endotracheal tube with respect to the bite tube in the axial direction,
wherein a lateral opening is formed along an entire length of the bite tube, the front plate and the adjustable retainer for lateral or transversal introduction of at least one endotracheal tube into the passage zone defined by the bite tube, and
wherein the front plate and the adjustable retainer are arranged externally with respect to the bite tube.

2. The device as claimed in claim 1, wherein the means of immobilisation of the endotracheal tube comprise a pressure clamp attached to the front plate open towards the lateral opening of the device and arranged in parallel to an exterior face of the front plate.

3. The device as claimed in claim 2, wherein the pressure clamp comprises an end part attached to the front plate and two elastically flexible arms with toothed concave portions, facing each other, to secure at least one endotracheal tube in the passage zone of the bite tube.

4. The device as claimed in claim 3, wherein the front plate comprises stops for securing the arms elastically bent into an open position of the pressure clamp.

5. The device as claimed in claim 1, wherein the adjustable retainer is constituted by an inflatable balloon.
